(19) United States
(12) Patent Application Publication
(10) Pub. No.: US 2026/0152781 A1
(43) Pub. Date: Jun. 4, 2026

(54) MECHANICAL HOMOGENIZATION

(71) Applicant: OMNI INTERNATIONAL, INC., Kennesaw, GA (US)

(72) Inventors: Zachary MOREHOUSE, Tampa, FL (US); Caleb PROCTOR, Acworth, GA (US); Rodney NASH, Tucker, GA (US)

(21) Appl. No.: 18/965,440

(22) Filed: Dec. 2, 2024

Related U.S. Application Data
(63) Continuation of application No. 17/330,928, filed on May 26, 2021, now abandoned.
(60) Provisional application No. 63/029,805, filed on May 26, 2020.
(60) Provisional application No. 63/064,447, filed on Aug. 12, 2020.

(51) Int. Cl. C12Q 1/6806; C12Q 1/70

(52) U.S. Cl. CPC C12Q 1/6806; C12Q 1/701

(57) ABSTRACT

Methods and systems of mechanically homogenizing an unprocessed sample are disclosed, that include a mechanical homogenizer that is used to mechanically homogenize the unprocessed sample to produce a homogenized sample. Nucleic acids in the homogenized sample are amplified without performing any isolation, separation, purification, or extraction of the nucleic acids. The amplification results are then analyzed to detect viral nucleic acids and/or the presence or absence of a virus in the sample.

### TECHNICAL FIELD

The present invention relates generally to mechanical homogenization, and particularly to use of mechanical homogenizers to facilitate the analysis of unprocessed biological samples.

### BACKGROUND

Viral nucleic acids from infected tissue, serological, and fluid samples are commonly used in genetic and molecular biology for identifying the presence of viral infections such as COVID-19. To obtain the viral nucleic acids, viral-infected cells of a biological sample are lysed to break down the sample cells, and then the viral nucleic acids (e.g., RNA, DNA) are extracted from the lysate. Conventional techniques for viral sample lyses include enzymatic digestion or vortexing, and conventional techniques for viral nucleic acid extraction from the resulting lysate include the use of extraction kits (e.g., magnetic binding, spin column kits, phenol-chloroform extractions, and TRIzol extractions) or a series of chemical washes resulting in isolation and purification of the viral nucleic acids. As such, viral nucleic acid isolation, purification, and/or extraction are separate steps after viral lysing, and the combined processes are time-consuming and require special reagents. The result is that the overall viral detection process is time-consuming and sub-optimally efficient. At the same time, viral infections are on the rise globally, especially given the current COVID-19 pandemic, and increased viral nucleotide based testing has become of a critical importance.

### SUMMARY

Generally described, the present disclosure relates to mechanically homogenizing unprocessed samples. According to the disclosed methods and systems, the unprocessed samples are mechanically homogenized to produce a homogenized sample. Then nucleic acids in the homogenized sample are amplified, without performing any isolation, separation, purification, or extraction of the nucleic acids in the homogenized sample. And then the amplification results are analyzed to detect viral nucleic acids and/or the presence or absence of a virus in the sample. Mechanically homogenizing the unprocessed sample can be done for example using a bead mill homogenizer. The unprocessed sample can be for example a respiratory tract sample or another biological sample.

In some embodiments, the unprocessed sample is a biological sample obtained in a homogenizer-ready tube. As an example, the unprocessed biological sample can be obtained on a swab contained in a homogenizer-ready tube. In such embodiments, the homogenizer-ready tube containing the biological sample can be loaded into a laboratory homogenizer, without transferring the biological sample to a separate tube for the homogenizing.

In some embodiments, mechanically homogenizing the unprocessed sample includes homogenizing the unprocessed sample at an oscillatory rate of about 0.8 m/s to about 8.0 m/s. In some embodiments, homogenizing the unprocessed sample includes subjecting the unprocessed sample to g-forces of about 10 g to about 450 g.

In one aspect, the unprocessed sample includes at least one of: cells infected with a virus, and viral particles. As such, mechanically homogenizing the unprocessed sample can include lysing the unprocessed sample so that at least seventy percent of the cells and the viral particles in the homogenized sample are lysed.

In some embodiments, the amplifying includes performing PCR on viral nucleic acids in the homogenized sample. Also, the amplifying can include amplifying at least one of DNA or RNA associated with a virus.

Further disclosed is diluting a respiratory tract sample before homogenizing the respiratory tract sample. Also, the respiratory tract sample can include a saliva sample. Further, the amplifying can includes amplifying at least one of DNA or RNA associated with a respiratory virus.

The specific techniques and structures employed to improve over the drawbacks of the prior art and accomplish the advantages described herein will become apparent from the following detailed description of example embodiments and the appended drawings and claims.

### BRIEF DESCRIPTION OF THE DRAWINGS

FIG. 1 is a process flow diagram of a method of processing biological samples for direct viral nucleic acid amplification according to an example embodiment.

FIG. 2 is a Cq graph showing a comparison of test results of conventional preprocessing and amplification methods and the direct amplification method of FIG. 1 for cotton swabs spiked with HCoV-229E and held in viral transport media.

FIG. 3 is a Cq graph showing a comparison of test results of conventional preprocessing and amplification methods and the direct amplification method of FIG. 1 for cotton swabs spiked with HCoV-229E and held in viral transport media.

FIG. 4 is a Cq graph showing test results of the direct amplification method of FIG. 1 for cotton swabs spiked with influenza A virus and held in viral transport media.

FIG. 5 is an amplicon gel visualization of the results on the test results of FIG. 4.

FIG. 6 is a Cq graph showing a comparison of test results of conventional preprocessing and amplification methods and the direct amplification method of FIG. 1 for cotton swabs spiked with influenza A virus and held in viral transport media.

FIG. 7 is an amplicon gel visualization of the results on the test results of FIG. 4.

### DETAILED DESCRIPTION OF EXAMPLE EMBODIMENTS

Generally described, the present disclosure relates to methods of analyzing viral nucleic acids (RNA and DNA) in a biological sample (from a human or other animal) using a mechanical homogenizer, thereby exposing the viral nucleic acids in the resulting homogenized sample, and thereafter, without any isolation, separation, purification, or extraction of the (viral nucleic acids in the) homogenized sample, proceeding directly to amplifying the viral nucleic acids in the homogenized sample. Furthermore, the biological sample submitted to the mechanical homogenizer is unprocessed, and as used herein, “unprocessed” with reference to a biological sample (“sample”) shall mean a biological sample that has not been treated with a lysis reagent (e.g., a detergent or surfactant) or subjected to vortexing; however, as provided herein, an unprocessed sample shall be understood to include, in some embodiments, a sample that has optionally been diluted with a suitable diluent. Thus, the mechanical homogenization of the unprocessed sample results in lysis and exposing/freeing of viral nucleic acids from cells and/or viral capsids, without the need for using lysis reagents, enzymatic digestion, or vortexing before the mechanical homogenization, and without any isolation, separation, purification, and/or extraction of the viral nucleic acids after the mechanical homogenization but before amplifying the viral nucleic acids. The mechanical homogenization is thus comparable or more effective for lysis than other lysis methods such as those using lysis reagents, vortexing, and/or enzymatic digestion, and the mechanical homogenization is sufficiently effective for exposing viral nucleic acids for detection via standard molecular biology applications such as amplification, without the need for isolating, purifying, and/or extracting the viral nucleic acids (e.g., DNA and/or RNA), and/or without the need for a protease. As used herein, the term “homogenized sample” means a biological sample that has been subjected to a mechanical homogenizer for sufficient time to lyse at least seventy percent (70%) of the cells and/or viral particles in the biological sample.

This one-step mechanical lysis of an unprocessed sample using a mechanical homogenizer without any isolation, purification, or extraction of the nucleic acids in the sample prior to amplification thereof, can be used for a range of different types of biological samples from humans (or other animals). In example embodiments, the biological sample is a respiratory tract biological material, e.g., from the mouth, nose, throat, or lungs. Such respiratory tract biological materials include saliva, mucus, sputum, lavages (e.g., bronchoalveolar lavages), and/or other secretions from the respiratory tract, and thus also include nasopharyngeal swabs and oropharyngeal swabs with the sample collected on them. After subjecting the unprocessed sample to a mechanical homogenizer, the homogenized sample is directly (i.e., without any processing of the homogenized sample, such as the addition of a protease, or isolation, purification, or extraction of the viral nucleic acids) presented to an amplifier of the viral nucleic acids and to thereafter for detection of the viral nucleic acids/respiratory virus infection, including coronaviruses (e.g., SARS-COV-2), influenza (e.g., A, B, or C), rhinoviruses, and respiratory syncytial virus (RSV), paramyxoviruses (e.g., measles, mumps, parainfluenza). In other example embodiments, the biological sample is another biological material, such as tissue, blood, urine, feces, and/or other biological material. In such embodiments, the homogenized sample may contain a greater amount of amplification (e.g., PCR) inhibitors as compared to other sample, such that an optional dilution of the sample prior to mechanical homogenization, may be desired.

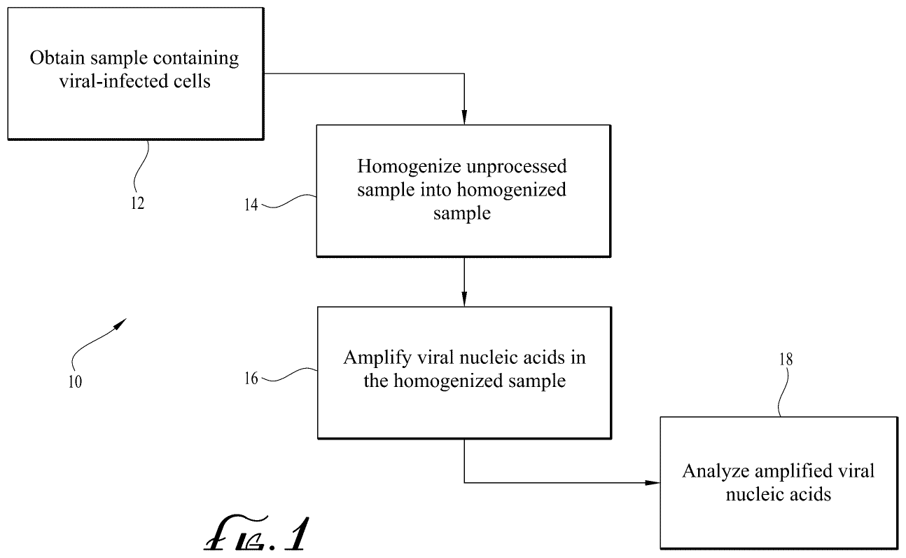

Referring now to the drawings, FIG. 1 shows a method 10 of analyzing biological samples for direct viral nucleic acid amplification according to an example embodiment. The method includes obtaining a biological sample suspected of containing cells infected by a virus or body fluids containing viral particles at 12, mechanically homogenizing the unprocessed sample to create a homogenized sample that includes exposed viral nucleic acids (e.g., DNA, RNA) at 14, amplifying the viral nucleic acids in the homogenized sample at 16 without performing any extraction, isolation, separation, and/or purification of the viral nucleic acids in the homogenized sample, and thereafter, analyzing the amplification results to detect the viral nucleic acids and/or the presence or absence of the virus at 18 in the biological sample.

In some embodiments, the homogenizing 14 and the amplifying 16 are performed at the same location by the same laboratory for efficiency. In other embodiments, the homogenizing 14 and the amplifying 16 are performed at different locations by the same or different laboratories.

The sample collection step 10 can be performed in various ways, including collecting the sample on a swab or collecting the sample directly into a tube or other conventional sample container. Direct-collection methods include collecting the sample (e.g., expelled saliva or drawn blood) directly into a tube, and other conventional methods that do not include collecting the sample on a swab and placing the sample-laden swab in a tube.

The sample type selection may consider whether the suspected-infected cells in the sample are fast growing, as such condition may be preferable in some embodiments. In embodiments, a sample type may be selected to be one that has few natural PCR inhibitors. Those of ordinary skill will also understand that certain samples, once collected, may be diluted prior to mechanical homogenization at 14 using a suitable diluent, for example, if the sample contains greater numbers of natural PCR inhibitors compared to samples that contain lesser numbers of such inhibitors. In some embodiments, the sample may contain viral particles instead of and/or in addition to infected cells, and the disclosed methods and systems may operate on such sample in the same manner as described herein. Such samples may also be optionally diluted prior to the mechanical homogenization.

For swab-collected sampling methods, the swabs can be conventional swabs (e.g., nasopharyngeal swabs, oropharyngeal swabs, nasal swabs, buccal swabs, or rectal swabs) and the sample-laden swab is placed into a tube with a viral transport media (VTM) for storage and transport until it can be mechanically homogenized. In example embodiments, the sample-laden swab is placed into a mechanical homogenizer-ready tube containing a VTM for storage, transport, and homogenizing. The mechanical homogenizer-ready tubes can also be preloaded with beads or other grinding media for use on a bead-mill homogenizer. That is, the tube containing the sample-laden swab and VTM is sealed, transported to a location (e.g., a laboratory) and homogenized, without breaking the seal on the tube. The sample-laden swab does not need to be removed from the tube and/or processed in any way before proceeding to mechanical homogenization 14.

For example, the sample-bearing swab can be dropped, sample-end first/down, directly into a homogenizer-ready tube containing the VTM. In typical embodiments, the swab shaft is longer than the homogenizer-ready tube. For example, conventional 2 mL homogenizer-ready tubes (OMNI International, Inc.) are about 4 cm long and conventional nasopharyngeal swabs are significantly longer. With the sample end of the swab inside the tube, the non-sample end of the shaft protruding from the tube is then broken off, the tube cap is placed over the open end of the tube and secured (e.g., screwed) on to close the tube, and the closed tube containing the sample-laden swab is sealed and transported to the laboratory (or other location with a mechanical homogenizer). At the laboratory, the tube holding the sample-bearing swab and VTM can be loaded into the mechanical homogenizer for mechanical lysing and exposing at 14 without any additional swab handling.

This method has proven effective for respiratory tract biological specimens that are mucus, sputum, and/or cellularly derived. Saliva typically includes significant amounts of PCR inhibitors, but also typically includes significant amounts of virus. Saliva samples can be diluted with an appropriate diluent (e.g., with PBS, normal saline, nuclease free water, and/or VTM) and still be analyzed according to the disclosed methods 10. The dilution can be done at the collection site or at the laboratory before the mechanical homogenization 14.

For direct-collection methods, the sample can be collected directly into a mechanical homogenizer-ready tube containing a VTM for storage, transport, and homogenizing. The mechanical homogenizer-ready tubes can also be preloaded with beads or other grinding media for use on a bead-mill homogenizer, and the sample can also be diluted (as noted above). That is, the tube containing the sample and VTM is sealed, transported to a location (e.g., a laboratory) and homogenized, without breaking the seal on the tube. The sample does not need to be removed from the tube and/or processed in any way before proceeding to mechanical homogenization 14.

Suitable example homogenizer-ready tubes for this purpose include homogenizer tubes available from OMNI INTERNATIONAL, Inc. (Kennesaw, Georgia), such as OMNI 1.5, mL, 2 mL, and 7 mL homogenizer tubes with screw caps. As used herein, a “homogenizer-ready tube” is a tube or well plate that can be placed into a laboratory homogenizer and used during mechanical homogenization of the sample contained within it, so these tubes are very heavy duty and can withstand the extremely high forces created by the homogenizer during the mechanical homogenization. The size of the homogenizer-ready tube can be selected based on the homogenizer type and manufacturer to be used, as will be understood by persons or ordinary skill in the art. In some embodiments, the homogenizer used in the homogenizing 14 can be selected or adapted to use homogenizer-ready tubes sized to contain an intact conventional nasopharyngeal or oropharyngeal swab without removing any of the non-sample end of the swab shaft.

Suitable example VTMs for this purpose include universal transport media (UTM), phosphate buffered saline (PBS), Hanks balanced salt solution (HBSS), other balanced salt solutions, genetic material preserving solutions, lysing buffers, or extraction buffers. Of these, UTM is believed to be particularly well suited for sample storage, transport, and homogenizing. The VTM used can be selected based on the sample, tube, homogenizer, and virus for a particular application, as will be understood by persons or ordinary skill in the art.

In other example swab-collection methods, the sample-laden swab can be dropped into a conventional sample-collection tube (e.g., 15 mL or 12 mL) containing a VTM, which is then transported to the laboratory. Before homogenizing at 14, the sample-laden swab is first transferred from the collection tube into a homogenizer-ready tube containing a VTM. Other than transferring the sample-laden swab to a homogenizer-ready tube, no other processing of the sample-laden swab is needed before homogenizing at 14.

The sample can be collected, e.g., from a patient by a swab at a point-of-care location, a field-testing location, or elsewhere. The tube containing the sample-laden swab and VTM can be transported frozen, refrigerated, or at room temperature to the laboratory for analysis. If frozen (e.g., at −20 C to −80 C for storage and/or transport for extended time periods), the sample-laden swab is thawed prior to homogenizing at step 14.

Next, at 14, the biological sample is mechanically homogenized to provide a homogenized sample. More particularly, the homogenization 14 mechanically lyses the cells and/or the viral particles to release the viral nucleic acids in the sample cells and/or the viral particles/capsids so that the viral nucleic acids are free floating in the resulting homogenized sample. The inventors surprisingly found that the intensity of the mechanical homogenization on an unprocessed sample could be ready for direct use (i.e., without any isolation, separation, purification, or extraction of the viral nucleic acids in the homogenized sample) in viral nucleic acid amplification.

The homogenization 14 can be performed using a conventional mechanical homogenizer. Homogenization involves disaggregating, mixing, re-suspension, or emulsifying the components of a sample using high shear with significant micron-level particle-size reduction of the sample components. Because very large forces must be generated for homogenization, the unprocessed sample is subjected to very high oscillatory rates. The present disclosure thus contemplates the use of any type of mechanical homogenizer.

In example embodiments, the homogenization 14 is performed using a shaker-mill homogenizer (aka a bead-mill homogenizer) such as the BEAD RUPTOR ELITE homogenizer (OMNI INTERNATIONAL, Inc., Cat. No. 109-141E). This mechanical homogenizer includes a hub to which a processing plate is removably mounted, with the hub inducing a vigorous “swashing” motion of the processing plate, and with the processing plate holding tubes containing the samples to be homogenized by the vigorous swashing motion. This swashing motion of the processing plate and tubes is not rotational about the center of the processing plate, but instead is angularly reciprocating to induce a force with a rotational (sinusoidal) component and an axial component, and can be described as a sigmoidal or oscillatory function.

In example embodiments, the homogenizer and tube are selected so that the reciprocating component of the homogenization motion is greater/longer than the length of the sample tube, though in other embodiments it can be less than the tube length. It should be noted that other types and brands of homogenizers can be used in the homogenization 14, including those producing a purely linear reciprocating motion.

The homogenization 14 includes operating the homogenizer at very high oscillatory rates to generate very high g-forces to produce the homogenized sample. For example, the homogenizer can be operated at oscillatory rates of about 0.8 m/s to about 8.0 m/s, with a speed of about 4.2 m/s for about 30 s having been shown to produce satisfactory results when including beads in the tubes. Persons of ordinary skill in the art will appreciate that the homogenizer can be operated at other speeds and for other time periods to produce the homogenized sample.

For example, good results have been obtained by homogenizing at velocities from about 3.1 m/s to about 6.0 m/s and durations from about 15 s to about 60 s and receiving a positive RT-qPCR result confirming the presence of virus and successful viral lysis. Also, for speeds of about 0.8 m/s to about 8.0 m/s, the homogenization 14 has been determined to produce g-forces from about 10 g to about 450 g at each direction change in the oscillatory motion. Furthermore, homogenizing at a speed of about 4.2 m/s for about 15 s has been shown to produce an average lysis of about 62.5%, at about 4.2 m/s for about 30 s has been shown to produce an average lysis of about 97.0%, and at about 4.2 m/s for about 15 s has been shown to produce an average lysis of about 87.5%. As used herein, the homogenized sample can be considered to have been lysed to at least about 70% of the cells and/or viral particles, to at 80% lysis preferably, and to at least about 90% most preferably. In some embodiments, the percent lysis is at least about 70%, at least about 75%, at least about 80%, at least about 85%, at least about 90%, or at least about 95% of the cells and/or viral particles.

In addition, for purposes of this disclosure, a shaker-mill homogenizer can be a bead-mill homogenizer operated without any beads or other grinding media. The disclosed methods can be performed with or without beads (e.g., ceramic or metal) or other grinding/beating media in the tube. Higher forces are transferred to the sample when beads are included in the tubes. The homogenizer can be operated at lower speeds and/or for shorter time periods when beads are used as compared to when beads are not used. Those of ordinary skill will thus understand that the present disclosure is not limited by the type of mechanical homogenizer or the manner by which the mechanical homogenizer performs its homogenization to achieve the desired lysis of the unprocessed sample.

At this point, the homogenized sample is ready to directly proceed to amplification 16. This, there is no need to extract, purify, separate, or isolate the viral nucleic acids from the homogenized sample, or otherwise perform any other procedure with or to the homogenized sample, before amplification of the viral nucleic acids (e.g., RNA and/or DNA associated with the virus) in the homogenized sample. Accordingly, the homogenized sample produced by the mechanical homogenizer is taken directly to amplification at 16, without any extraction, separation, isolation, purification, etc.

The amplification 16 can include conventional nucleic acid detection techniques such as conventional polymerase chain reaction (PCR) techniques and machines. These can include, for example, RT-qPCR (reverse transcription, quantitative PCR), RT-PCR (reverse transcription PCR), endpoint PCR, multiplex PCR, and rPCR (rapid PCR). Other amplification techniques and machines can be used, for example rolling circle replication/amplification, sequencing reaction amplification, loop-mediated isothermal amplification (LAMP), or any other type of amplification. Those of ordinary skill will thus understand that the present disclosure is not limited to the amplification technique used to amplify the suspected viral nucleic acid.

Finally, the amplified nucleic acids are analyzed at 18 to determine the presence of viral nucleic acids (e.g., RNA and/or DNA associated with the virus). This can be done by, e.g., conventional visualization procedures such as amplicon visualization using agarose gel, or other known nucleic acid detection techniques, and the present disclosure is not limited to the type of nucleic acid detection technique employed.

### Experimental Procedures

The direct-amplification method 10 was used in tests to determine its efficacy. In each of the following experiments, samples were taken using sterile cotton nasopharyngeal swabs (Cat. No. 22-029-488) by FISHER SCIENTIFIC COMPANY LLC and homogenizer-ready tubes (Cat. No. 19-648) by OMNI INTERNATIONAL, Inc. The swabs were placed sample-end first into 2 mL homogenizer-ready tubes containing 1 mL of VTM, the non-sample ends were broken off, and the tube caps were screwed on to seal the tubes closed. The tubes containing the virally spiked swabs and the VTM were then homogenized (as detailed in the results section below) before amplification using RT-qPCR (as detailed in the results section below).

These experiments and the resulting data are discussed in two publications, The Utility of Mechanical Homogenization in COVID-19 Diagnostic Workflows, by Morehouse, Nash, Proctor, and Ryan (IntechOpen, Mar. 30, 2021), and A Novel Two-step, Direct-to-PCR Method for Virus Detection off Swabs using Human Coronavirus 229E, by Morehouse, Nash, Ryan, and Proctor (Virology Journal, Aug. 25, 2020), incorporated herein by reference in their entirety.

### Experimental Data Results 1: Human Coronavirus 229E

In these tests, human coronavirus 229E was used as the model system. For the RT-qPCR, the HCoV-229E nucleocapsid gene (N gene) was selected as the target, based on the clinical diagnostic PCR target recommendations from the US Center for Disease Control and Prevention. The N gene was targeted with forward primer 5′-AGGCGCAAGAATTCAGAACCAGAG-3′ reverse 5′- and primer AGCAGGACTCTGATTACGAGAAAG-3′. 1 uL of the homogenized sample was added to create a final reaction volume of 20 uL using the proportions of primers, RNA, SYBER, RT, and DPEC laid out in the New England Biologics Luna RT-qPCR Kit (NEB, Cat. No. E3005S). The reaction was run on a BIORAD CFX Connect Real-Time PCR Detection System with the CFX MAESTRO software package (BIORAD, Cat. No. 1855200) for 44 cycles, and the resulting amplicons were loaded into a 2% agarose gel for product visualization.

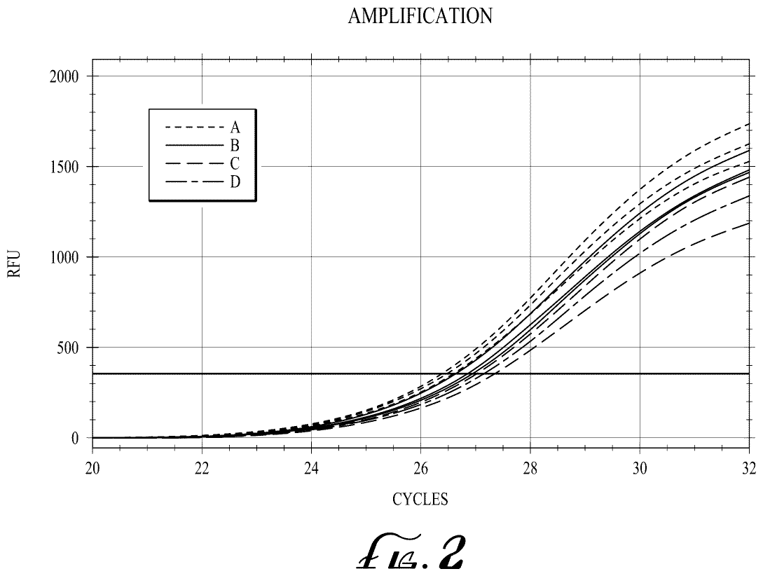

FIG. 2 shows a comparison of three sample techniques using cotton swabs spiked to clinical levels of HCOV-229E and held in VTM. The curved lines A show results for homogenizing the homogenizer-ready tubes containing the swab head and VTM on the BEAD RUPTOR ELITE homogenizer (OMNI INTERNATIONAL, Inc., Cat. No. 109-141E) for 30 s at 4.2 m/s, then removing 1 uL of the homogenized sample (after the froth settled), and then proceeding directly to amplification of the homogenized sample using RT-qPCR. The curved lines B show results for processing with a vortexer and then taking 1 uL of the lysate directly to RT-qPCR. The curved lines C show results for no processing methods and then taking 1 uL of the sample media directly to RT-qPCR. And the curved line D shows results for a swab spiked with a non-virally infected sample and placed into a homogenizer-ready tube (as described above) and run on the OMNI INTERNATIONAL BEAD RUPTOR ELITE homogenizer for 30 s at 4.2 m/s and then taking 1 uL of the sample media directly to RT-qPCR.

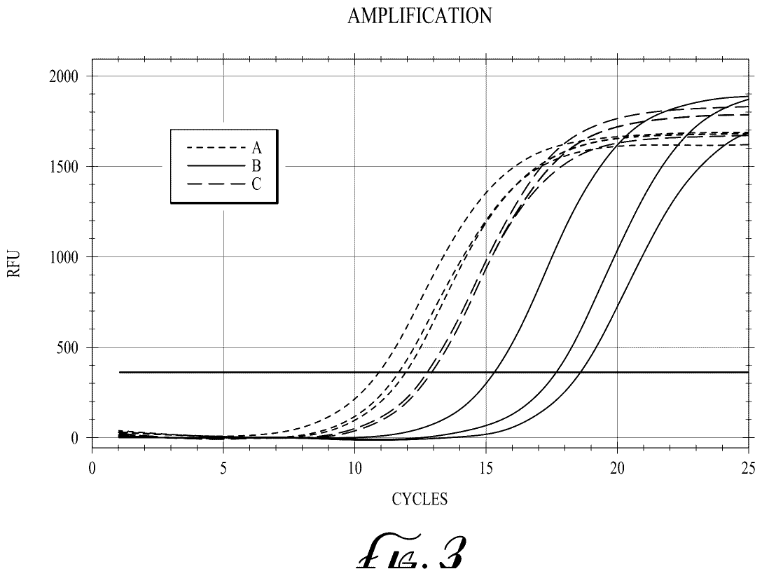

FIG. 3 shows viral RNA extraction off of spiked swab samples using RNA extraction kits to compare efficacy of conventional sample processing methods and the direct-amplification method 10. This figure shows the RT-qPCR results following the comparison of three techniques prior to the use of the same RNA extraction kit used to clean up the resulting genetic products. The curved lines A show results for homogenizing on the OMNI INTERNATIONAL BEAD RUPTOR ELITE homogenizer for 30 s and 4.2 m/s, the curved lines B show results for enzymatic digestion using a commercial viral lysis buffer for sample processing, and the curved lines C show results for vortex processing. All nine samples then underwent extraction using an OMNI INTERNATIONAL RNA extraction kit following the viral RNA extraction protocol and the resulting RNA was used for RT-qPCR to determine abundance of RNA obtained from each sample in the extraction process.

The PCR inhibitors present in the sample media used for RT-qPCR cause the visualization of the product amplification to be narrowed (as seen in FIG. 1) due to the reactions needing to overcome the inhibition threshold. However, using the data generated and displayed in FIG. 1 in conjunction with the data shown in FIG. 2, we can extrapolate the sample pattern illustrating that both with and without the use of genetic extraction kits, homogenization using the OMNI INTERNATIONAL BEAD RUPTOR ELITE homogenizer provides increased yield of extracted viral nucleic acids in comparison to both vortex and enzymatic digestion procedures currently employed in viral genetic extraction protocols.

The results of these experiments demonstrate detection of human coronavirus 229E RNA via direct RT-qPCR from the homogenized sample. In particular, we have proven viral detection via nucleic acid amplification across a range of viral load levels spiked onto swabs. We have proven positive results off swabs spiked with 106 to 103 PFU/mL (plaque forming units/mL). This range of viral load has been correlated with the average viral load range seen on clinical swabs as reported by Y. Pan et al. in The Lancet in February 2020.

### Experimental Data Results 2: Influenza A Virus

In these tests, influenza A virus (IAV) was used as the model system. Supernatant was collected from IAV-infected MDCK sample cells and non-infected MDCK sample cells. 500 uL of supernatant was transferred to an OMNI 19-628 homogenizer-ready bead tube and homogenized as detailed above (on the OMNI BEAD RUPTOR ELITE at 4.2 m/s for 30 s). For the RTqPCR, 1 ul of the homogenized sample was added directly to RTqPCR reaction mix containing IAV primers for M gene. The samples were allowed to amplify for 45 cycles. Amplicons were visualized on 2% agarose gel stained with EtBR and imaged using a gel documentation system. The primers were obtained from a Spackman matrix test and ordered from Integrated DNA Technologies, Inc.

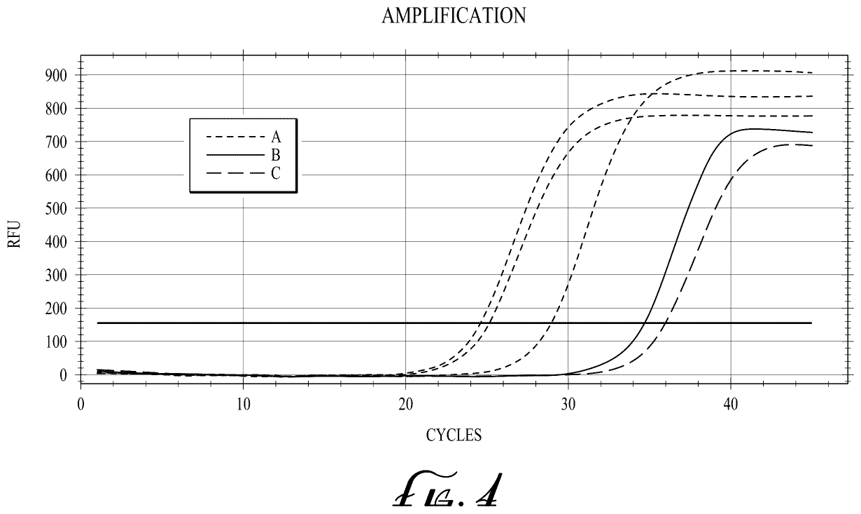

FIG. 4 shows a comparison of three techniques using cotton swabs spiked to clinical levels of IAV and held in VTM. The curved lines A show results for homogenizing the homogenizer-ready tubes containing the swab head and VTM on the OMNI INTERNATIONAL BEAD RUPTOR ELITE homogenizer for 30 s at 4.2 m/s, then removing 1 uL of the homogenized sample (after the froth settled), and then proceeding directly to amplification of the homogenized sample using RT-qPCR. The curved line B shows results for a swab spiked with a non-virally infected sample and placed into a homogenizer-ready tube (as described above) and homogenized on the OMNI INTERNATIONAL BEAD RUPTOR ELITE homogenizer for 30 s at 4.2 m/s and then taking 1 uL of the sample media directly to RT-qPCR. And the curved line C shows results for a DEPC negative control test.

The Cq information is provided below in Table 1, which provides an average Cq value of 26.18.

TABLE 1

| Well | Fluor | Target | Content | Sample | Cq |
|---|---|---|---|---|---|
| A01 | SYBR | M gene | Unknown | IAV 1 | 25.07 |
| A02 | SYBR | M gene | Unknown | IAV 2 | 24.50 |
| A03 | SYBR | M gene | Unknown | IAV 3 | 28.97 |
| A04 | SYBR | M gene | Unknown | Non-infected | 34.63 |
| A05 | SYBR | | Unknown | DEPC | 36.00 |

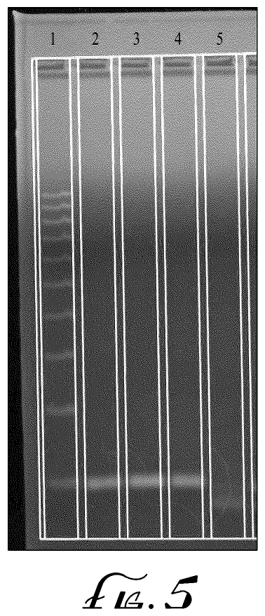

FIG. 5 (lanes 1-5) shows the amplicon visualization for the sample testing of FIG. 4 (samples A-B) and Table 1 (wells A01-A05). The M gene product is visualized at 100 bp (lane 1). The primer dimers are seen <100 bp in the non-infected sample (lane 5/sample B/well A05). The three tests for the IAV-spiked swabs are shown to detect the presence of IAV (lanes 2-4/sample A/wells A01-A03).

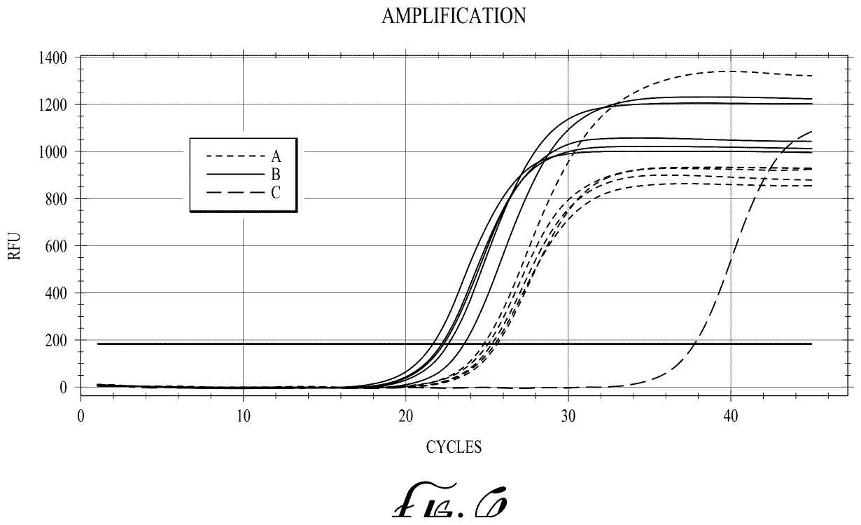

FIG. 6 shows a comparison of three techniques using cotton swabs spiked to clinical levels of IAV and held in VTM. The curved lines A show results for homogenizing the homogenizer-ready tubes containing the swab head and VTM on the OMNI INTERNATIONAL BEAD RUPTOR ELITE homogenizer for 30 s at 4.2 m/s, then removing 1 uL of the homogenized sample (after the froth settled), and then proceeding directly to amplification of the homogenized sample using RT-qPCR. The curved lines B show results for the same homogenizing and amplifying but additionally including RNA extraction using an OMNI INTERNATIONAL RNA extraction kit for sample processing before amplifying. And the curved line C show results for a DEPC negative control test.

The Cq information is provided below in Table 2, which provides an average Cq value of 25.26 (SD 0.3145267) for the direct amplification procedure (lines A of FIGS. 6) and 22.46 (SD 0.7155743) for the same but with extraction added (lines B of FIG. 6).

TABLE 2

| Well | Fluor | Target | Content | Sample | Cq |
|---|---|---|---|---|---|
| A01 | SYBR | M gene | Unknown | IAV extract 1 | 23.56 |
| A02 | SYBR | M gene | Unknown | IAV extract 2 | 22.62 |
| A03 | SYBR | M gene | Unknown | IAV extract 3 | 22.31 |
| A04 | SYBR | M gene | Unknown | IAV extract 4 | 21.61 |
| A05 | SYBR | M gene | Unknown | IAV extract 5 | 22.19 |
| A06 | SYBR | M gene | Unknown | IAV direct 1 | 25..05 |
| A07 | SYBR | M gene | Unknown | IAV direct 2 | 25.63 |
| A08 | SYBR | M gene | Unknown | IAV direct 3 | 25.29 |
| B01 | SYBR | M gene | Unknown | IAV direct 4 | 25.46 |
| B02 | SYBR | M gene | Unknown | IAV direct 5 | 24.85 |
| B03 | SYBR | | Neg. control | DEPC | 37.73 |

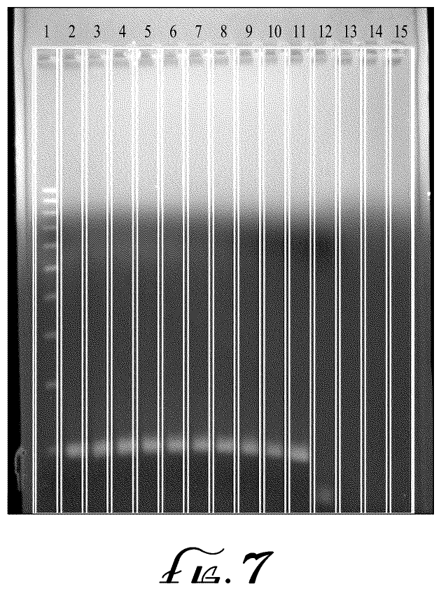

FIG. 7 (lanes 1-12) shows the amplicon visualization for the sample testing of FIG. 6 (samples A-C) and Table 2 (wells A01-B03). The M gene product is visualized at 100 bp (lane 1). The primer dimers are seen <100 bp in the control sample (lane 12/sample C/well B03). The last five lanes (lanes 7-11) were not used in this experiment. The five tests for the direct-amplification samples (lanes 7-11/samples A/wells A06-B02) show this technique to be comparably effective in detecting the presence of IAV to the five tests that included extraction (lanes 2-6/samples B/wells A01-A05).

The results of these experiments demonstrate detection of IAV RNA via direct RT-qPCR from the homogenized sample. In particular, FIGS. 4-5 show that the direct PDR method 10 can be used to detect IAV RNA, and FIGS. 6-7 show the efficacy of the direct PDR method to detect IAV RNA as compared to conventional methods.

Accordingly, advantageous aspects and uniqueness of various embodiments include the following:
1. Direct-to-PCR: Viral nucleic acid detection from an unprocessed sample using mechanical homogenization and amplification alone, without lysis reagents, vortexers, buffers, purification, isolation, separation, and/or extraction kits, or, leads to increased efficiency and lower cost.
2. Higher throughput: Processing multiple samples simultaneously using multi-sample homogenizers (e.g., 24 sample tubes per machine) allows the user to overcome bottlenecks with current testing approaches by increasing the number of samples handled at once, thereby decreasing the total processing time for a batch of samples. Current protocols are focused on the processing of one patient sample at a time.
3. Higher throughput: Individual sample processing times using homogenization are much quicker (e.g., 30 seconds of shaker-mill homogenization) than current protocols (e.g., 60 seconds of vortexing).
4. Increased safety for laboratory personnel: Patient samples can be placed into the homogenizer in a sealed homogenizer-ready tube, thereby lessening worker contact with open viral samples. Currently, patient samples are manually transferred by a lab technician from the initial collection tube to a separate processing tube or plate for lysing. This increases the potential of contact with the sample and an increased need for personal protective equipment to be used in the laboratory.

It is to be understood that this invention is not limited to the specific devices, methods, conditions, and/or parameters described and/or shown herein, and that the terminology used herein is for the purpose of describing particular embodiments by way of example only. Thus, the terminology is intended to be broadly construed and is not intended to be limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been shown and described in exemplary forms, it will be apparent to those skilled in the art that many modifications, additions, and deletions can be made therein without departing from the spirit and scope of the invention as defined by the following claims.

1. A method of mechanically homogenizing an unprocessed sample, the method comprising:
mechanically homogenizing the unprocessed sample to produce a homogenized sample;
amplifying nucleic acids in the homogenized sample without performing any isolation, separation, purification, or extraction of the nucleic acids; and,
analyzing the amplification results to detect viral nucleic acids and/or the presence or absence of a virus in the sample.

2. The method of claim 1, further comprising obtaining the unprocessed sample in a homogenizer-ready tube.

3. The method of claim 2, wherein the unprocessed sample is a respiratory tract sample.

4. The method of claim 2, wherein obtaining the unprocessed sample includes obtaining a biological sample on a swab contained in a homogenizer-ready tube.

5. The method of claim 4, wherein mechanically homogenizing the unprocessed sample includes loading the homogenizer-ready tube containing the biological sample into a laboratory homogenizer, without transferring the biological sample to a separate tube for the homogenizing.

6. The method of claim 1, wherein mechanically homogenizing the unprocessed sample includes homogenizing the unprocessed sample at an oscillatory rate of about 0.8 m/s to about 8.0 m/s.

7. The method of claim 1, wherein homogenizing the unprocessed sample includes subjecting the unprocessed sample to g-forces of about 10 g to about 450 g.

8. The method of claim 1, where the unprocessed sample comprises at least one of: cells infected with a virus, and viral particles.

9. The method of claim 8, wherein mechanically homogenizing the unprocessed sample includes lysing the unprocessed sample so that at least seventy percent of the cells and the viral particles in the homogenized sample are lysed.

10. The method of claim 1, wherein mechanically homogenizing the unprocessed sample includes using a bead mill homogenizer.

11. The method of claim 1, wherein the amplifying includes performing PCR on viral nucleic acids in the homogenized sample.

12. The method of claim 1, wherein the amplifying includes amplifying at least one of DNA or RNA associated with a virus.

13. The method of claim 1, wherein the sample is a respiratory tract sample.

14. The method of claim 13, further including diluting the respiratory tract sample before homogenizing the respiratory tract sample.

15. The method of claim 13, wherein the respiratory tract sample includes a saliva sample.

16. The method of claim 13, wherein the amplifying includes amplifying at least one of DNA or RNA associated with a respiratory virus.
